(19) United States
(12) Patent Application Publication
(10) Pub. No.: US 2017/0135311 A1
(43) Pub. Date: May 18, 2017

(54) ANIMAL CONTAINMENT SYSTEM

(71) Applicant: MDC Exports Limited, Bedfordshire (GB)

(72) Inventor: Melvyn Brian Driver, Bedfordshire (GB)

(21) Appl. No.: 15/312,749

(22) PCT Filed: May 18, 2015
(86) PCT No.: PCT/GB2015/051450

§ 371 Date: Nov. 21, 2016

(30) Foreign Application Priority Data
May 20, 2014 (GB) 1408929.6
Jun. 11, 2014 (GB) 1410365.9
Nov. 19, 2014 (GB) 1420569.4

(51) Int. Cl. A01K 1/03; A61G 10/04; A61M 16/10; A01K 1/00; A61D 7/00

(52) U.S. Cl. CPC A01K 1/031; A01K 1/0064; A61D 7/00; A61M 16/1005; A61G 10/04; A61M 2202/0208

(57) ABSTRACT

An apparatus comprising an animal containment part with one or more openings, a cover part for entirely surrounding the containment part, said cover part being flexible and transparent and including means for entry of a gaseous therapeutic agent.

### CROSS-REFERENCE TO RELATED APPLICATION

This application is a national phase filing under 35 U.S.C. §371 based on International Application No. PCT/GB2015/051450, having an International filling date of May 18, 2015, for an “APPARATUS,” the disclosure of which is hereby incorporated by reference.

### BACKGROUND OF THE INVENTION

The present invention relates to an animal containment system, and in particular to one which is for use in a therapeutic treatment of an animal.

Oxygen is one of the most common agents administered in veterinary practices, and is essential in the intensive care setting. There are a variety of non-invasive and invasive methods of delivering oxygen therapy to an animal. With regard to non-invasive methods, commonly used oxygen delivery means are masks, hoods and incubators. Masks and hoods are often not tolerated well by animals and incubators are extremely expensive pieces of apparatus and are usually restricted to specialist referral units and University departments. Determining the most suitable means of oxygen therapy can be a challenging task and tolerance of each method varies from patient to patient. Oxygen administration should never be stressful for the patient. If the animal becomes anxious or frightened, and starts to struggle, an alternative method should be initiated.

CA2437258 discloses a method of inducing pulmonary dilation of a bird with gaseous nitric oxide, which method includes the steps of providing at least one bird in a cage, draping a piece of material over the cage and delivering a predetermined concentration of gaseous nitric oxide into the draped cage.

US2007/272166 discloses a system for respiratory pharmacology testing and a chamber device that enables respiratory pharmacology testing on particular large animals under un-anaesthetized, unrestrained state, with easy acclimatization and reduced effect of stress, and a method for conducting safety pharmacology studies on the same large animal individual. The chamber device of the invention includes an animal cage and a transparent chamber, which includes first and second compartments separated by a partition plate. The first compartment has a door and a ventilation connection port, and the first and second compartments are provided with a communication pipe having an opening for introducing ambient air. The device has a ventilation connection port in a side face of the chamber, and a communication pipe for introducing air on the top face of the chamber, so that, with the cage placed in the compartment, air flow through the compartment is maintained constantly and allowed to communicate through the cage.

US2009/159011 discloses a small animal veterinary incubator with an animal holding drawer that can be inserted into a temperature controlled enclosure and removed when not in use for cleaning and disinfecting. In various embodiments, the incubator is portable, is at least partly transparent, controls interior oxygen and/or humidity, can use a hose to bring oxygen into close proximity with incubated animals, can introduce water-nebulized air or oxygen into the drawer through a hole, and can include a holder for a humidity bottle that humidifies air or oxygen supplied to the incubator. Some embodiments use an adjustable or regulated lamp to warm the incubator using an infrared bulb. Temperature, humidity, and/or oxygen gages or sensors can be included, and the temperature, oxygen, and/or humidity can be automatically regulated.

U.S. Pat. No. 4,941,431 discloses a system for treating laboratory animals with a gaseous medium while the animals are in a laboratory cage. The system includes a limited number of lids adapted to fit the entire range of standard laboratory plastics cages. The lids are in three sizes, each size designed to co-operate with a different group of the cages. The system includes means to supply a gaseous medium to one or more of the lids and includes controls having predetermined settings to regulate the rate of flow and the period of flow of the gaseous medium to the lids to provide a volume of medium selected according to the size of the cage to obtain efficacious treatment of the animals.

EP1234540 discloses a method and apparatus for readily and reliably screening olfactory mucosa stimulating compounds. An olfactory mucosa stimulating compound is sprayed through an atomizing nozzle to an olfactory mucosa of a rat fixed in a test animal fixing device. A measuring electrode portion is implanted in an olfactory bulb of the rat for measuring an electrical signal generated in the olfactory bulb. Efficacy of the olfactory mucosa stimulating compound is determined based on a correlation between an electrical signal measured by the measuring electrode portion when the olfactory mucosa stimulating compound is sprayed on the olfactory mucosa of the rat and a physiological response induced in the rat.

### SUMMARY OF THE INVENTION

According to the present invention, there is provided an apparatus comprising an animal containment part with one or more openings, a cover part for entirely surrounding the containment part, said cover part being flexible and transparent and including means for entry of a gaseous therapeutic agent.

Owing to this invention, an animal containment system can be provided to deliver relatively good overall fractional oxygen concentration to the lungs of an animal, which system can be tolerated by the animal and which is not an expensive piece of apparatus. In addition, the cover part which covers the entire containment part is able to retain the gaseous therapeutic agent without significant leakage.

In a preferred embodiment, the animal containment part is an animal cage with at least a plurality of walls and a lid being of a metallic mesh and thus having a plurality of openings to the surrounding atmosphere. The cover part fits over the entire containment part and includes at least one fastening device, such as a zip, in order to cover all of the openings in the containment part. The means for entry can be the fastening device or a separate closable opening of the cover part. Thus, a tube from an oxygen source can then be used to deliver an amount of therapeutic oxygen whilst the fastening device is still not completely fastened. Once sufficient oxygen has been delivered into the containment system, the tube can be withdrawn and the fastening device fully closed. Advantageously, the fastening device traverses over the top portion of the cover part owing to the fact that oxygen is heavier than air and so will sink to the lower region of the containment part, which then acts as a reservoir, and which is, in the case of relatively small animals and/or unwell animals, where the animal will be located.

The apparatus is also an advantageous route for administering pharmaceuticals in a nebulized form, particularly for small animals for which masks are extremely impractical. The gaseous mist of the pharmaceutical compound is simply sprayed into the animal containment part and contained therein by the cover part and inhaled by the animal.

In order that the present invention can be clearly and completely disclosed, reference will now be made, by way of example only, to the accompanying drawing. In the drawing, like reference numerals refer to like parts through the several views, in which:

### BRIEF DESCRIPTION OF THE DRAWING

FIG. 1 is a perspective view of an animal containment system; and

FIG. 2. is a side elevation view of the animal containment system.

### DETAILED DESCRIPTION OF THE INVENTION

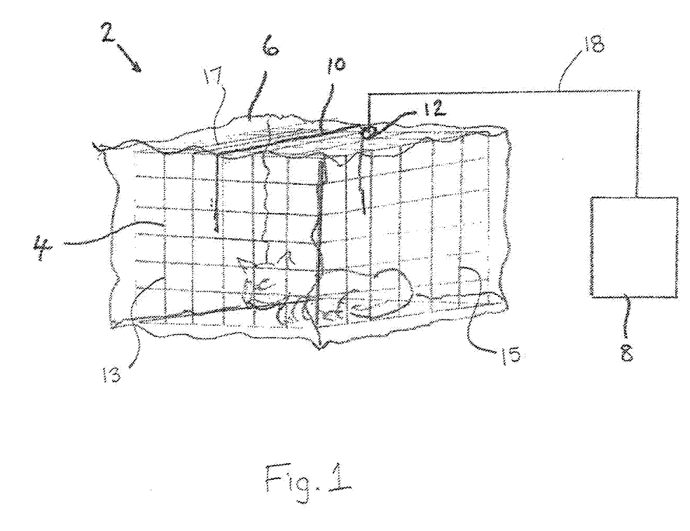

Now, and in accordance with the present invention and with reference to FIG. 1, there is provided an animal containment system 2. The animal containment system 2 comprises a containment part 4, a cover part 6 and a source of a gaseous therapeutic agent 8, such as oxygen. The containment part 4 is in the form of a cage with at least a plurality of side walls 13, 15 and a lid 17 being made from a steel mesh, which may or may not be plastics coated. The cover part 6 is preferably made from a flexible transparent plastics material and which includes at least one fastening device 10, such as a zip in order to surround the containment part 4. The source of therapeutic oxygen 8 can then be arranged to deliver oxygen via a tube 20 into the containment system 2 by way of means of entry 12 in the cover part 6. The means of entry 12 can be the fastening device 10 or some other closable opening in the cover part 6. The means of entry 12 is preferably located in the top region of the cover part 6 because oxygen is heavier than air and thus will sink towards the bottom region of the containment part 4, where the animal being treated will be located because it is a small animal and/or it is a sick animal which will naturally be spending most of its time in a laying-down position in a tray substantially the same size as and resting upon the floor of the containment part 4.

A pharmaceutical in a nebulized or mist form can also be introduced into the containment part 4. This is particularly advantageous when small animals are being treated owing to the fact that it is very difficult to get such small animals to wear a conventional mask for delivery of such pharmaceuticals. In this instance, once the nebulized pharmaceutical is injected or sprayed through the means of entry 12, the gaseous mist is held within the confines of the containment part 4 to be inhaled by the animal.

The cover part 6 is preferably made from a transparent plastics material in order to be able to visually inspect the animal patient and any equipment placed inside the containment part 4 as part of the animal's treatment, such as climate control devices which measure temperature and/or humidity inside the system 2. If humidification is not provided, the patient will be likely to experience drying and dehydration of the nasal mucosa, respiratory epithelial degeneration, impaired mucociliary clearance and an increased risk of infection. The cover part 6 is advantageously made from polyvinyl chloride (PVC) and of a suitable thickness, which is substantially 3 mm. Any seams of the cover part 6 and the edges where it joins with the fastening device 10 may be heat-sealed and/or stitched in order to make sufficiently good seals to prevent unwanted leakage of the gaseous therapeutic agent.

Conventional animal enclosures are known to comprise contact surfaces of plastics material, which may be solid plastics or plastics-coated wire. Such enclosures are common in transporting animals, particularly domestic pets, to a Veterinary Practice. However, from the perspective of Veterinary Practitioners, these known enclosures can be responsible for the transfer of pests and parasites from one animal to another. When an infected animal is placed in such an enclosure, the pest or parasite can remain in the enclosure which may then be used, subsequently, to transport another animal which would then have a high risk of contracting the pest or parasite. The preferred method to sterilise the enclosure and thus eradicate the pest or parasite is flame treatment (the use of chemical agents is not suitable owing to possible effect on an animal to be placed in the enclosure). Flame treatment is not suitable for a plastics contact surface.

Thus, the preferred containment part 4 has animal contact surfaces being substantially of a heat resistant material. In this way, containment part 4 can be thoroughly sterilised by flame treatment. Advantageously, the material is stainless steel. Stainless steel has the advantage, from the point of view of the containment part 4, that it is a strong material and is rust-resistant. If pests or parasites are deposited onto these metallic surfaces they can be efficiently eradicated by flame treatment.

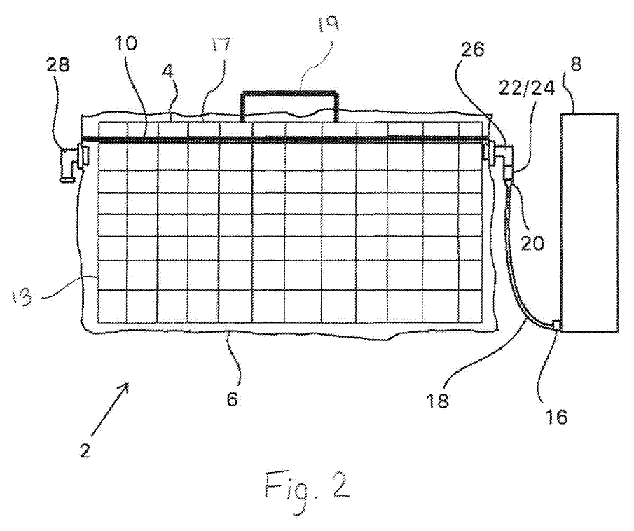

The containment part 4 could therefore be substantially free from plastics materials, except perhaps for a small part such as a carrying handle 19 (shown in FIG. 2). A range of colors can be used and the advantage of such range of colors is that, in a veterinary practice, veterinary surgeons and/or veterinary nurses prefer to arrange the containment parts 4 according to a color coding system which may relate to the type of treatment required or administered, or some other preferred arrangement.

As is known, stainless steel is resistant to corrosion, staining and needs very little maintenance. It is also of relatively low cost with a familiar lustre. In addition, stainless steel has its own antibacterial properties and does not need paint or other surface finishes. Furthermore, the passivation layer formed by the chromium in the alloy is too thin to be visible, and the metal remains lustrous whilst protecting the metal beneath. This passivation layer quickly reforms when the surface is scratched. This passivation of the alloy is seen in other metals which could also be used, such as aluminium and titanium.

Now, with reference to FIG. 2, advantageously, the containment system 2 is provided with an accessory pack containing all the necessary equipment required to simply then connect the system 2 to the source of the gaseous therapeutic agent 8. This equipment comprises a first connector 16 for connecting to the source 8, the tube 18 for conducting the gaseous agent, a second connector 20, a nebulizer 22, at least one venturi device 24, and a coupling device 26 to couple with the cover part 6 of the system 2. In use, one end of the tube 18 is connected to the first connector 16, which is connectable to the source 8. The opposite end of the tube 18 can either be connected to the nebulizer or to the venturi device 24. The nebulizer 22 or the venturi device 24 can be connected to the second connector 20, which in turn is connected to the coupling device 26. The coupling device 26 is preferably in the form of an elbow coupler with a flange at its free end opposite to the end connected to the second connector 20, which flange is of a larger diameter than the means for entry 12, which is advantageously in the form of a simple circular opening in the upper region of the cover part 6. Immediately downstream of the flange is a first washer fitted over a threaded section which passes through the means for entry 12, with the first washer located on the outside of the cover part 6. An oppositely threaded nut is then screwed onto the threaded section inside the cover part 6 preferably together with a second washer such that the wall of the cover part 6 surrounding the means for entry 12 is sandwiched between the first and second washers. This forms a sufficiently good seal around the means for entry 12 to prevent any significant leakage. The area of the cover part 6 around the means for entry 12 is preferably thicker than the remainder of the cover part in order to provide extra stability in that region for repeated connection of the coupling device 26. This could for instance simply be a double layer of the material from which the cover part 6 is made.

Preferably, there are a plurality of venturi devices 24 supplied in the accessory pack. When carrying out oxygen therapy, the oxygen from the source 8 will flow through the tube and into the venturi device 24 at which point atmospheric air is drawn in and mixed with the oxygen. The plurality of venturi devices 24 have different sized openings in order to produce different mixtures of oxygen and air. The venturi devices 24 are ideally color coded in accordance with the mixture produced. The resultant ratio of air in the mixture is consistent for each venturi device 24 regardless of the oxygen flow rate. When deciding which venturi device 24 to use, oxygen requirements vary depending on the particular aliment being treated. For example, an animal with fractured ribs only needs around 24-28% oxygen in the mixture, whilst an animal with congestive heart failure requires around 60% oxygen in the mixture. Each of the venturi devices 24 delivers a different amount of oxygen and the correct one needs choosing, which is why color coding of the venturi devices 24 is beneficial.

In conditions where the alveolar gas exchange mechanism is unaffected or where there is a mechanical or physiological impairment of ventilation, such as with a ruptured diaphragm or rib fractures, a venturi device 24 that allows 24-28% oxygen in the mixture would be the correct one to choose. Where the alveolar gas exchange mechanism is impaired, in such conditions as chronic bronchitis or a mild pulmonary contusion, a venturi device 24 that allows 35-40% oxygen in the mixture would be the correct one to choose. Where the alveolar gas exchange mechanism is severely impaired in more serious conditions such as Bronchopneumonia, severe pulmonary contusion or congestive heart failure, a venturi device 24 that allows 60% oxygen in the mixture would be the correct one to choose. With the system 2 of dimensions of approximately 46 cm×30 cm×30 cm, the volume of such system is approximately 42 liters. Once the correct venturi device 24 has been selected, the minute tidal volume requires calculation and this is 10 ml/kg×breaths per minute. In order to ensure that adequate air/oxygen mixture is replenished into the system 2, an in-flow of around twice the minute tidal volume should be aimed for.

A second coupling device 28 could also be provided as a spare or to couple with a second means for entry in the cover part 6 to enable the introduction of another gaseous medium, such as an anesthetic.

One of the problems with conventional expensive incubators is that many do not provide climate control to adjust temperature and/or humidity. Animal patients can rapidly become hypothermic, and humidity increases over time, which means such devices should only be used for short periods in order to avoid the above-mentioned complications. Similarly, if climate control is not to be used with the containment system 2, then the system should only be used for short periods of time, with the advantage that the system 2 is much less costly than an incubator.

In order to clinically assess patients in the system 2, the system 2 will have to be opened, resulting in a decrease in oxygen concentration inside the system 2 (if oxygen therapy is taking place). Depending on the nature of the condition of the patient, this may reduce the fractional oxygen concentration to the level of atmospheric oxygen, with the end result being an inability to accurately control oxygen concentrations but the treatment will still be highly beneficial to the patient.

Venting of the system will naturally take place through small openings in the cover part 6 where the fastening device is located and if a separate means for entry 12 is provided.

Monitoring equipment should always be used to evaluate the animal's response to the oxygen treatment, along with careful observation and clinical examination. Ideally, arterial blood gas analysis should be performed to provide a clinical picture of oxygen delivery to the tissues.

The system 2 provides a cost-effective and relatively stress-free method of oxygen therapy for animals. It is also ideal for emergency situations and allows veterinary professionals to provide a therapeutic agent single-handedly.

Furthermore, the containment part 4 may be supplied with a removable lid section which can facilitate the taking of x-rays of the animal. Clearly, in order to obtain a reliable x-ray, the animal must be still and is more likely to be settled in the containment part 4 rather than having to remove the animal for x-ray. The cassette is then placed under the tray in the containment part 4 and the x-ray equipment can then be arranged directly over the open-topped containment part 4 for the image to be captured.

Having thus described the invention, what is claimed is:

1. An apparatus comprising: an animal containment part with one or more openings, a cover part for entirely covering surrounding the containment part, said cover part being flexible and transparent and including means for entry of a gaseous therapeutic agent.

2. The apparatus according to claim 1, wherein the animal containment part is an animal cage having a plurality of walls and a lid disposed atop the plurality of walls, the plurality of walls and the lid being of a metallic mesh and thus having a plurality of openings to the surrounding atmosphere.

3. The apparatus according to claim 1, wherein the animal containment part is a steel containment part.

4. The apparatus according to claim 1, wherein the animal containment part comprises is a stainless steel containment part.

5. The apparatus according to any preceding claim 1, wherein the cover part includes at least one fastening device.

6. The apparatus according to claim 5, wherein the means for entry is the fastening device.

7. The apparatus according to claim 1, wherein the means for entry is a separate closable opening of the cover part.

8. The apparatus according to claim 5, wherein the fastening device traverses over the a top portion of the cover part.

9. The apparatus according to claim 1, wherein the gaseous therapeutic agent is oxygen from an oxygen source.

10. The apparatus according to claim 1, wherein the gaseous therapeutic agent is a nebulized form of pharmaceutical.

11. The apparatus according to claim 1, wherein the containment part includes a removable lid.

12. The apparatus according to claim 1, and further comprising an accessory pack containing equipment required to connect the apparatus to a source of the gaseous therapeutic agent.

13. The apparatus according to claim 12, wherein the equipment comprises a first connector for connecting to the source, a tube for conducting the gaseous agent, a second connector, a nebulizer, at least one venturi device and a coupling device to couple with the cover part.
